(19) United States
(12) Patent Application Publication
(10) Pub. No.: US 2004/0096484 A1
(43) Pub. Date: May 20, 2004

(54) Antioxidant-containing wipes and absorbent products for improving skin health

(76) Inventors: David J. Tyrrell, Appleton, WI (US); Beth A. Lange, Appleton, WI (US); Duane G. Krzysik, Appleton, WI (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/299,156

(22) Filed: Nov. 19, 2002

(51) Int. Cl. A61K 35/80; A61K 9/70; A61K 35/78

(52) U.S. Cl. 424/443; 424/729; 424/732; 424/195.17; 424/765; 424/764; 424/766; 424/736; 424/737

(57) ABSTRACT

Disclosed are methods products such as wet wipes and absorbents capable of providing a skin health benefit when utilized in the intended fashion. More specifically, the products described herein comprise an antioxidant agent, such as a botanical extract, which is capable of reducing the amount of oxidation on the skin's surface resulting in improved skin health. The antioxidant agent has an antioxidant potential at three minutes of at least about 25% as determined by a AST Antioxidant Status Test set forth herein. In another embodiment, the antioxidant agent has an antioxidant potential at thirty minutes of at least about 20% as determined by a AST Antioxidant Status Test set forth herein.

### BACKGROUND OF THE INVENTION

The present invention relates to personal care products such as wet wipes and absorbent products capable of providing a skin health benefit. More particularly, the present invention relates to wet wipes comprising an antioxidant agent, such as a botanical extract, capable of acting as an antioxidant on or near the surface of the skin to reduce skin oxidation and improve skin health. The compounds described herein and suitable for incorporation into a wet wipe solution include botanical extracts having an antioxidant potential at three minutes of at least about 25% according to a AST Antioxidant Status Test. When incorporated into the wet wipe solution, the antioxidant agents described herein improve the overall mildness of the solution by reducing the oxidative effects to the skin of the surfactants contained therein.

It is well known that skin can easily be damaged through exposure to solar radiation, environmental pollutants, chemical compounds, as well as through contact with biological insults such as feces. Some of the damage incurred by the skin through such exposure is a direct result of oxidation on the skin's surface; that is, the direct result of the interaction of free radicals (atoms or molecules with unpaired electrons) such as oxygen, nitrogen, and carbon centered radicals with the skin. Reactive oxygen species produced from the oxygen molecule include singlet oxygen, the superoxide anion radical, hydrogen peroxide, and the hydroxyl radical, as well as reaction products produced by these free radicals which can interact with, and severely damage, the skin through various oxidation reactions.

Almost every cellular constituent is a potential free radical target. The main targets of oxidation in the epidermis are the proteins and the lipids. Approximately 25% of the total skin surface lipids are unsaturated. Polyunsaturated lipids are vulnerable to oxidation due to the presence of bis allylic hydrogens, which are prone to hydrogen abstraction. This lipid peroxidation can cause membrane disruption or ultimately give rise to compounds that are mutagenic or sensitizers.

Aging and drying of the skin can occur when collagen becomes hard and meshes with neighboring collagen fibers. This interaction prevents the collagen from holding water and plumping, and the collagen actually begins to collapse on itself, binds with other collagen fibers, and forms a fishnet structure below the surface of the skin. As human tissue is bombarded with external insults that include ultraviolet radiation, a cascade of free radical formation takes place, leading to often severe alteration in tissue structure. The overall net effect is indiscriminate cross-linking of collagen fibers, which can be revealed on the skin's surface as wrinkles and dry skin. Further, reactive oxygen species such as those described above can react with other molecules besides collagen (often with unsaturated lipids in the epidermis) and generate a cascade of harmful free radical reactions in the skin.

Along with environmental factors and body exudates, other factors can contribute to the formation of free radicals and the oxidation of skin. For example, some components of commonly utilized consumer products may cause oxidation of the skin and result in damage. The liquid formulation of wet wipes, which are commonly utilized to clean body exudates from both adult and infant skin, typically contain at least one surfactant, to promote skin cleansing. Surfactants however, even of the mild type, can induce oxidation and other biochemical processes in and on the skin which can lead to redness, edema, and irritation. Because surfactants are typically a required component of the liquid formulations used with the wet wipes, the induction of such pro-inflammatory events is almost a certainty in some individuals, particularly those individuals classified with “sensitive skin.”

Based on the foregoing, there is a need for products such as wet wipes that can reduce the potential for oxidative damage to the skin from environmental factors, contact with various bodily exudates, and commercial products. Commercially economical absorbent products and wet wipes comprising an antioxidant agent, which is mild on the skin yet provides significant antioxidant potential would be beneficial and could result in improved skin health through a reduction of oxidation-induced skin damage.

### SUMMARY OF THE INVENTION

The present invention relates to products and methods for improving skin health by reducing oxidative-induced skin damage. More specifically, the present invention relates to products such as wet wipes and absorbent articles containing agents, which are capable of improving the health of skin they contact during normal usage by reducing the amount of oxidative-induced skin damage resulting therefrom of the surface of skin.

In one embodiment of the present invention, the mildness of wet wipe solutions can be significantly improved by introducing an antioxidant agent directly into the liquid wet wipe solution or formulation. The antioxidant agent may have an antioxidant potential at three minutes of at least about 25% as determined by a AST Antioxidant Status Test. Alternatively, the antioxidant agent may have an antioxidant potential at thirty minutes of at least about 20% as determined by a AST Antioxidant Status Test. By introducing an antioxidant agent, such as a botanical extract, into the wet wipe solution, the mildness of the solution is improved as the solution is less likely to impart oxidative damage to the skin. In a preferred embodiment, the antioxidant agent introduced into the wet wipe solution is substantially active for at least about thirty minutes; that is, the antioxidant agent has an antioxidant potential of at least about 25% at three minutes and at least about 20% at thirty minutes.

Briefly, therefore, the present invention is directed to a wipe for improving skin health comprising a fibrous wipe substrate and an antioxidant agent for reducing oxidative-induced skin damage. The antioxidant agent has an antioxidant potential at three minutes of at least about 25%.

The present invention is further directed to a wipe for improving skin health comprising a fibrous wipe substrate and an antioxidant agent for reducing oxidation on the surface of the skin. The antioxidant agent has an antioxidant potential at thirty minutes of at least about 20%.

The present invention is further directed to a wipe for improving skin health comprising a fibrous wipe substrate and an antioxidant agent for reducing oxidative-induced skin damage. The antioxidant agent has an antioxidant potential at three minutes of at least about 75% and an antioxidant potential at thirty minutes of at least about 75%.

The present invention is further directed to a method for controlling oxidative-induced skin damage to improve skin health. The method comprises contacting a fibrous wipe substrate with the skin surface. The fibrous wipe substrate comprising an antioxidant agent having an antioxidant potential at three minutes of at least about 25%.

Other objects and features of this invention will be in part apparent and in part pointed out hereinafter.

### DETAILED DESCRIPTION OF THE PREFERRED EMBODIMENTS

In accordance with the present invention, it has been discovered that antioxidant agents, such as certain botanical extracts, can be utilized in common personal care products, such as wet wipes and absorbent articles, to maintain and improve skin health by reducing the amount of oxidative-induced skin damage. By reducing the oxidative potential of a wet wipe solution, the amount of skin damage is reduced. Some of the antioxidant agents described herein have antioxidant potentials at three minutes of at least about 25% as determined by a AST Antioxidant Status Test, other agents described herein have antioxidant potentials at thirty minutes of at least about 20% as determined by a AST Antioxidant Status Test, and still others posses both characteristics. By introducing an antioxidant agent having one or both of these antioxidant potentials into a wet wipe solution, the amount of oxidation caused by the surfactants present in the solution, or caused by other external factors, may be significantly reduced.

The personal care products of the present invention contain at least one antioxidant agent, such as a botanical extract, which is capable of improving the health of skin contacted by the personal care product during normal use by reducing the oxidative potential of the product. Numerous personal care products can be used with the antioxidant agents described herein in accordance with the present invention to improve the skin health of the user. For example, one or more of the antioxidant agents described herein can be used in combination with wipes, such as wet wipes, hand wipes, face wipes, cosmetic wipes, household wipes, industrial wipes, dry wipes and the like, to improve the health of the skin by reducing the amount of oxidation on the skin's surface. As will be understood by one skilled in the art, the antioxidant agent may be directly on the wipe substrate, or may be in a liquid formulation used with the wipe.

Materials suitable for the substrate of the wipe are well known to those skilled in the art, and are typically made from a fibrous sheet material, which may be either woven or nonwoven. For example, the wipes incorporating the additives described herein to improve skin health may include nonwoven fibrous sheet materials, which include meltblown, coform, air-laid, bonded-carded web materials, hydroentangled materials, and combinations thereof. Such materials can be comprised of synthetic or natural fibers, or a combination thereof. Typically, wipes define a basis weight of from about 25 to about 120 grams per square meter and desirably from about 40 to about 90 grams per square meter.

In a particular embodiment, the wipes incorporating the additives described herein comprise a coform basesheet of polymeric microfibers and cellulosic fibers having a basis weight of from about 60 to about 80 grams per square meter and desirably about 75 grams per square meter. Such coform basesheets are manufactured generally as described in U.S. Pat. No. 4,100,324, which is incorporated by reference. Typically, such coform basesheets comprise a gas-formed matrix of thermoplastic polymeric meltblown microfibers, such as, for example, polypropylene microfibers, and cellulosic fibers, such as, for example, wood pulp fibers.

The relative percentages of the polymeric microfibers and cellulosic fibers in the coform basesheet can vary over a wide range depending upon the desired characteristics of the wet wipes. For example, the coform basesheet may comprise from about 20 to about 100 weight percent, desirably from about 20 to about 60 weight percent, and more desirably from about 30 to about 40 weight percent of the polymeric microfibers based on the dry weight of the coform basesheet being used to provide the wipes.

Alternatively, the wipes incorporating the additives described herein can comprise a composite, which includes multiple layers of materials such as those described in U.S. Pat. No. 6,028,018, which is incorporated by reference. For example, the wipes may include a three layer composite, which includes an elastomeric film or meltblown layer between two coform layers as described above. In such a configuration, the coform layers may define a basis weight of from about 15 to about 30 grams per square meter and the elastomeric layer may include a film material such as a polyethylene metallocene film.

As mentioned above, one type of wipe suitable for use in combination with the additives described herein to improve skin health include wet wipes, which contain a liquid solution or formulation. The liquid can be any solution, which can be absorbed into the wet wipe basesheet and may include any suitable components, which provide the desired wiping properties. For example, the components may include water, emollients, surfactants, fragrances, preservatives, chelating agents, pH buffers, or combinations thereof as are well known to those skilled in the art. Further, the liquid may also contain lotions, medicaments, and/or antimicrobials.

The amount of liquid contained within each wet wipe may vary depending upon the type of material being used to provide the wet wipe, the type of liquid being used, the type of container being used to store the wet wipes, and the desired end use of the wet wipe. Generally, each wet wipe can contain from about 150 to about 600 weight percent and desirably from about 250 to about 450 weight percent liquid based on the dry weight of the wipe for improved wiping. In a particular aspect, the amount of liquid contained within the wet wipe is from about 300 to about 400 weight percent and desirably about 330 weight percent based on the dry weight of the wet wipe. If the amount of liquid is less than the above-identified ranges, the wet wipe may be too dry and may not adequately perform. If the amount of liquid is greater than the above-identified ranges, the wet wipe may be oversaturated and soggy and the liquid may pool in the bottom of the container.

Each wet wipe is generally rectangular in shape and may have any suitable unfolded width and length. For example, the wet wipe may have an unfolded length of from about 2.0 to about 80.0 centimeters and desirably from about 10.0 to about 25.0 centimeters and an unfolded width of from about 2.0 to about 80.0 centimeters and desirably from about 10.0 to about 25.0 centimeters. Typically, each individual wet wipe is arranged in a folded configuration and stacked one on top of the other to provide a stack of wet wipes. Such folded configurations are well known to those skilled in the art and include c-folded, z-folded, quarter-folded configurations and the like. The stack of folded wet wipes may be placed in the interior of a container, such as a plastic tub, to provide a package of wet wipes for eventual sale to the consumer. Alternatively, the wet wipes may include a continuous strip of material which has perforations between each wipe and which may be arranged in a stack or wound into a roll for dispensing.

In another embodiment, the personal care products which comprise one or more of the additives described herein for improving skin health can include absorbent personal care products such as diapers, training pants, adult incontinence garments, feminine napkins, paper towels, tampons, interlabial pads, facial tissue, wound management products, and bath tissue. Materials and methods for making such absorbent products are well known to those skilled in the art. When utilized in their intended manner, the absorbent personal care products including the antioxidant agent contact the skin such that the agent can reduce the amount of oxidative-induced skin damage and thus improve skin health.

In one embodiment of the present invention, an antioxidant agent or agents, such as certain botanical extracts, is introduced into or onto a personal care product to improve the health of skin contacted by the personal care product by reducing the amount of oxidative-induced skin damage. In a preferred embodiment, the antioxidant agent is introduced into the liquid formulation of a wet wipe to improve the mildness of the liquid formulation and counteract any skin oxidation that may occur due to the presence of one or more surfactants in the liquid formulation. As used herein, the term “mildness” refers to the amount of damage caused to the skin by a formulation, such as a wet wipe formulation and/or damage induced on the skin surface through wiping; that is, the more “mild” a formulation is, the less the damage or irritation imparted to the skin.

As mentioned above, surfactants are a common component in wet wipe solutions and can upregulate oxidative-induced cellular responses. By introducing the antioxidant agent into the wet wipe solution, the antioxidant agent can reduce the oxidative potential of the surfactant, reducing the level of oxidative-induced skin damage, and hence increase the mildness of the solution.

Preferably, the antioxidant agent, or combination of antioxidant agents, introduced into or onto the personal care product has an antioxidant potential at three minutes of at least about 25%, more preferably at least about 50%, and still more preferably at least about 75%. Further, in some embodiments, the antioxidant agent, or combination of antioxidant agents, introduced into or onto the personal care product has an antioxidant potential at thirty minutes of at least about 20%, more preferably at least about 50%, and still more preferably at least about 75%. As one skilled in the art will recognize, the higher the percentage of antioxidant potential, the more effective the antioxidant agent is at counteracting the oxidative potential of the solution. As such, it is typically preferred to have higher antioxidant potential compounds used with personal care products.

The antioxidant potential of the prospective antioxidant agent, at both three minutes and at thirty minutes, can be determined by numerous analytical methods known to those skilled in the art. A preferred analytical method for determining the antioxidant potential of a prospective antioxidant agent is a AST Antioxidant Status Test set forth below. This testing method can be utilized to determine an antioxidant agent's antioxidant potential at both three minutes and thirty minutes in accordance with the present invention. As one skilled in the art will recognize, other antioxidant screening tests could also be utilized in accordance with the present invention to evaluate compounds for antioxidant potential and use in the personal care products described herein.

AST Antioxidant Status Test

Potential antioxidant agents, such as liquid or solid botanical extracts and other compounds, for example, are analyzed for their antioxidant potential. The AST Antioxidant Status Test to determine the antioxidant potential of a potential antioxidant agent is completed as follows:

Step 1: A Total Antioxidant Status Kit from Randox Laboratories, Ltd. (United Kingdom) is obtained to measure antioxidant potentials of agents at three and thirty minutes.

Step 2: The potential antioxidant agent, if in liquid form, is diluted to a 1% (v/v) solution with deionized water. If the agent is in solid form, it is diluted to a 1% (w/v) solution with deionized water.

Step 3: From the Antioxidant Status Kit, the chromogen reagent is prepared for use by adding 10 milliliters of Phosphate Buffered Saline solution, pH=7.4, to a vial of chromogen 2.

Step 4: To the empty wells of a clear well plate, such as a NUNC IMMUNO clear 96 well plate (VWR Scientific Products, Chicago, Ill.) are introduced 5 microliters of the 1% testing solution prepared under Step 2 above and being analyzed for antioxidant potential. Control wells are also prepared wherein 5 microliters of deionized water are introduced in place of the 5 microliters of the 1% testing solution.

Step 5: To each well of the clear well plate containing the 5 microliters of 1% testing solution or 5 microliters of deionized water is added 250 microliters of chromogen reagent as prepared under Step 3 above.

Step 6: Optical density measurements of each well are taken at 600 nanometers without blanking the plate using a suitable microplate reader such as a SPECTRAmax PLUS 384 Microplate Reader (Molecular Devices, Sunnyvale, Calif.).

Step 7: The substrate provided in the Total Antioxidant Status Kit is prepared for use by diluting 1 milliliter of the substrate with 1.5 milliliters of Phosphate buffered Saline, pH=7.4.

Step 8: After an optical density reading was taken with 1% testing solution (or water) plus chromogen reagent, 50 microliters of the substrate prepared in Step 7 is added to each of the sample wells.

Step 9: After three minutes (or after thirty minutes if the antioxidant potential is to be determined for thirty minutes) the sample wells of the well plate are read at 600 nanometers without blanking the plate.

Step 10: The optical density readings measured are then utilized as follows to calculate the antioxidant potential (in percent) of the antioxidant agent:

% Antioxidant Potential=[(Δ_{ODWater}−Δ_{ODSample})/Δ_{ODWater})×100;

wherein

Δ_{ODWater}=OD_{Water}(Chromogen+Substrate+Water)−OD_{Water}(Chromogen+Water); and

Δ_{ODSample}=OD_{Sample}(Chromogen+Substrate+Agent)−OD_{Sample}(Chromogen+Agent)

Antioxidant agents for use in combination with the personal care products described herein preferably have high antioxidant potentials at both three minutes and at thirty minutes. Such a combination of antioxidant potentials means that the antioxidant agent is a strong antioxidant immediately upon use, and remains a strong antioxidant capable of maintaining its antioxidant activity for at least about thirty minutes. The thirty-minute time point is also important for detecting antioxidants where the rate reaction is slow such that they may appear as weak antioxidants at three minutes but provide scavenging of radicals over extended times. This is typically desirable in that the antioxidant agent can provide skin heath benefits to the user over an extended period of time.

Antioxidants typically react with an oxidant to form a more stable (less dangerous) end product. Traditional skin antioxidants such as vitamin C and vitamin E will react with radicals and in turn form free radicals themselves (ascorbly free radical and tocopheryl free radical). Antioxidants from botanicals and/or botanical extracts may provide a superior skin benefit because they contain a cocktail of antioxidant compounds to reduce the chance of an unstable end product forming.

Suitable antioxidant agents for use in combination with the personal care products described herein include antioxidant agents having an antioxidant potential at three minutes of at least about 25% as noted above. Specifically, it has been found that the following antioxidant agents have an antioxidant activity at three minutes of at least about 25% and are suitable for use in accordance with the present invention: Apple Green Tea, Arkin Special, Arnica Special, Avocado GW, Black Currant B, Black Currant Green Tea, Blueberry Fruit, Cabbage Rose Extract, Camellia sinensis, Canadian Willowherb, Carrot Root, Cemila Oleifera Extract, Common Thyme, Cranberry Green Tea, Echinacea Dry Aqueous Extract, Fennel Fruit, Ginko Biloba, glycine max (soybean seed), Goldenseal, Grapefruit, Grape Seed Extract and constituents thereof (i.e., proanthocyanidins), Grapefruit Green Tea, Green Tea, catechin constituents of green tea that include epigallocatechin gallate, epicatechin gallate, Green Tea Extra, Green Tea HS, Lime Blossom, Orange Green Tea, Phytexcell Arnica, Rosemary Plant, Sea Parsley, Secrets D' Orange, St. John's Wort W/S, Strawberry Fruit, Tomato Root, Turkish Oregano, Wheat Seed, White Mistle Toe, White Tea 50%, Yarrow, Yucca 70, Yucca Extract Powder (50%), and combinations thereof.

Suitable antioxidant agents for use in combination with the personal care products described herein to decrease oxidation on the skin's surface include antioxidant agents having an antioxidant potential at thirty minutes of at least about 20% as noted above. Specifically, it has been found that the following antioxidant agents have an antioxidant activity at thirty minutes of at least about 20% and are suitable for use in accordance with the present invention: Apple Green Tea, Canadian Willowherb, Cemila Oleifera Extract, Cranberry Green Tea, Echinacea Dry Aqueous Extract, Ginko Biloba, Grape Seed Extract, Grapefruit Green Tea, Green Tea, Green Tea Extra, Nab Willow Bark Extract, Orange Green Tea, White Tea 50%, Yucca 70, and combinations thereof.

When present on a dry wipe substrate or on an absorbent product such as a diaper, for example, the antioxidant agent that reduces oxidative-induced skin damage is present in an amount of from about 0.01% (by weight of the treated substrate or product) to about 50% (by weight of the treated substrate or product), preferably from about 0.01% (by weight of the treated substrate or product) to about 10% (by weight of the treated substrate or product). When present as part of the liquid formulation of a wet wipe, the antioxidant agent is present in an amount of from about 0.01% (by total weight of the liquid formulation) to about 50% (by total weight of the liquid formulation), more preferably from about 0.01% (by total weight of the liquid formulation) to about 10% (by total weight of the liquid formulation).

In another embodiment of the present invention, the personal care products described herein may contain a single antioxidant agent having a high antioxidant potential at both three minutes and at thirty minutes. In this embodiment, the antioxidant agent is highly effective in reducing the oxidative-induced skin damage and the resulting damage over an extended period of time. Since the majority of the oxidative damage from an insult happens in a matter of seconds, a potent antioxidant at 3 minutes that does not last substantially for 30 minutes in the AST would still be of significant value. Such an antioxidant agent having high antioxidant potential over an extended period of time may be particularly useful in wet wipe formulations which are typically utilized to remove bodily waste from the skin's surface. This type of product is generally utilized on the same area of skin numerous times daily on, for example, a baby. Due to the repeated contact of the skin with the surfactants contained in the formulation, skin damage may occur leading to red, irritated skin. By introducing an antioxidant agent having a high antioxidant potential for a period of at least about 30 minutes, skin damage can be minimized resulting in improved, healthy skin.

In this embodiment, it is preferred that the antioxidant agent have an antioxidant potential at three minutes of at least about 25%, more preferably at least about 50% and still more preferably at least about 75% and an antioxidant potential at thirty minutes of at least about 20%, more preferably at least about 50%, and still more preferably at least about 75% to impart the desired benefits. In accordance with these requirements, the following antioxidant agents have been found suitable: Apple Green Tea, Canadian Willowherb, Cemila Oleifera Extract, Cranberry Green Tea, Echinacea Dry Aqueous Extract, Ginko Biloba, Grape Seed Extract, Grapefruit Green Tea, Green Tea, Green Tea Extra, Orange Green Tea, White Tea 50%, and combinations thereof.

When present on a dry wipe substrate or on an absorbent product, the antioxidant agent having a high antioxidant potential for at least about 30 minutes for reducing oxidative-induced skin damage is present in an amount of from about 0.01% (by weight of the treated substrate or product) to about 50% (by weight of the treated substrate or product), preferably from about 0.01% (by weight of the treated substrate or product) to about 10% (by weight of the treated substrate or product). When present as part of the liquid formulation of a wet wipe, the antioxidant agent is present in an amount of from about 0.01% (by total weight of the liquid formulation) to about 50% (by total weight of the liquid formulation), more preferably from about 0.01% (by total weight of the liquid formulation) to about 10% (by total weight of the liquid formulation).

Liquid formulations comprising antioxidant agents such as botanicals described herein suitable for use in combination with a wipe substrate can be solutions, suspensions, or emulsions. Many of the antioxidant agents are substantially water-soluble or can be easily solubilized in water using techniques known to one skilled in the art to provide solutions comprising the antioxidant agent. Some antioxidant agents described herein which are not substantially water-soluble or easily solubilized can be suspended or emulsified utilizing techniques known to one skilled in the art. Suitable emulsions include oil-in-water emulsions which can be prepared using suitable emulsifiers having an HLB greater than about 7 or can be water-in-oil emulsions prepared using suitable emulsifiers having an HLB of less than about 7. In order to sufficiently protect the water-soluble and/or hydrophilic antioxidant agents from premature oxidation during processing and/or product shelf storage, water-in-oil emulsions are typically particularly useful. The emulsified layer around the water phase can prevent oxygen from entering the water phase.

Alternatively or additionally, the antioxidant agents can be encapsulated to prevent premature oxidation during process and product storage. Many encapsulation techniques known in the art can be utilized including those which provide sustained release, triggered release, targeted release or a combination of these release mechanisms. Other release mechanisms known in the art and suitable for use in accordance with the present invention include friction/pressure release, pH related release, water release, water evaporation release, and the like. Encapsulated antioxidant agents can be delivered from both wet and dry wipe products. Suitable microencapsulation materials suitable for use in combination with the antioxidant agents described herein include those available from Salvona, LLC (Dayton, N.J.).

Additionally, liposomes and/or nanosomes can be utilized to deliver and protect the antioxidant agents described herein from premature oxidation in both a wet and a dry wipe product. As used herein, the terms liposomes and nanosomes are meant to include closed vesicles with walls composed of lipid-bilayer membranes to protect and deliver the core antioxidant material.

Another method for delivering antioxidant agents from a wet or dry wipe product and protecting these components from premature oxidation includes utilizing polymeric entrapment systems such as microspheres, microsponges, and polytraps such as those available from Advance Polymer Systems (Redwood City, Calif.). The antioxidant agent can be entrapped or adsorbed into the interstitial spaces of a polymer matrix thereby protecting the antioxidant agent from degredation and/or premature oxidation.

Particularly useful for delivering the antioxidant agent from dry wipe substrates are semi-solid or solid formulations which transfer to the skin during use by the consumer. These formulations can be hydrophobic or hydrophilic in nature. The hydrophobic formulations are semi-solid or solid in nature at room temperature with a melting point greater than about 35° C. and contain from about 5 to about 95 weight percent of an emollient, from about 5 to about 95 weight percent of a solidifying agent such as an alpha olefin polymer, polyethylene, oxidized polyethylene, fatty alcohol, wax, or solid esters with a melting point of 35° C. or greater, from about 1 to about 50 weight percent of a viscosity enhancer such as silica, ethylene vinyl acetate copolymers, or an organo-clay and from about 0.1 to about 15 weight percent of an antioxidant agent.

The hydrophilic formulations are semi-solid or solid at room temperature with a melting point greater than about 35° C. and contain from about 30 to about 90 weight percent of a hydrophilic solvent/emollient such as water, propylene glycol, butylene glycol, low molecular weight (less than about 720) polyethylene glycols, dipropylene glycol glycerin, silicone glycols, methyl propanediol, or pentylene glycol, from about 10 to about 50 weight percent high molecular weight (greater than about 750) polyethylene glycol, from about 5 to about 40 weight percent of a fatty alcohol having from about 14 to about 30 carbons in the chain, optionally from about 1 to about 15 weight percent of a viscosity enhancer such as clays, glyceryl polyacrylates, or glyceryl polymethacrylates and from about 0.1 to about 15 weight percent of an antioxidant agent.

The dry wipe formulations can also be readily emulsifiable solid formulations with a melting point greater than about 35° C. and contain from about 5 to about 95 weight percent of a hydrophobic or hydrophilic emollient as set forth above, from about 5 to about 95 weight percent of a suitable solidifying agent as set forth above, from about 5 to about 25 weight percent of a suitable emulsifier and from about 1 to about 15 weight percent of an antioxidant agent. The suitable emulsifier can be anionic, cationic, amphoteric, zwitterionic, or nonioinic and combinations thereof.

In order to enhance consumer appeal, additional ingredients can be added to the above-described formulations. Suitable additional ingredients include, for example, anti-acne actives, antifoaming agents, antimicrobial actives, antifungal actives, antiseptic actives, antioxidants which prevent oxidation during processing and storage by preferentially oxidizing to preserve the antioxidency of the antioxidant agent described herein, astringents, colorants, deodorants, film formers, fragrances, moisturizers, skin protectants, sunscreen actives, and solvents.

Without being bound to a particular theory, it appears that the antioxidant agents described herein for reducing the amount of oxidative-induced skin damage accomplish this in one of two ways, or through a combination of both. First, the antioxidant agents, contained in the personal care products described herein, once present in or on the skin's surface may actually reduce the amount of highly reactive oxygen species produced that can directly or indirectly initiate events that lead to skin damage. For example, with wet wipe formulations, the antioxidant agent may reduce the oxidative-induced pathways turned on by the surfactant or other ingredients present in the wet wipe formulation that can lead to skin inflammation.

Alternatively or additionally, the antioxidant agents described herein may reduce the amount of oxidation on the skin's surface by actually serving as a substrate for reaction with highly reactive oxidative species that form on the skin's surface; that is, the antioxidant agents may provide a reactive substrate for the oxidative species to react with such that direct interaction with the skin molecules is minimized. This minimization of reactions with the skin may lead to less oxidative damage and improved skin health.

The present invention is illustrated by the following example which is merely for the purpose of illustration and is not to be regarded as limiting the scope of the invention or manner in which it may be practiced.

### EXAMPLE 1

In this Example, botanical extracts and other compounds were tested to determine their antioxidant potential at three minutes and at thirty minutes as determined by a AST Antioxidant Status Test.

Each of the compounds tested, the company where the compound was purchased, the city and state of the company, the lot number and catalog number of each compound (if available), and the total antioxidant potential at three and thirty minutes are set forth in Table 1.

TABLE 1

| | | | | % | % |
|---|---|---|---|---|---|
| | | | | Potential | Potential |
| Botanical | Company | Location | Lot#/Catalog# | @ 3 min. | @ 30 min. |
| Angelica Root PE 4:1 | Bio- | Hauppauge, | 921653/4012SD | 5.2 | 2.8 |
| | botanica | N.J. | | | |
| Apple Green Tea | Dragoco | Totowa, N.J. | 2/037050/L74247 | 95.3 | 42.1 |
| | | | 7 | | |
| Arkin Special | Dragoco | Totowa, N.J. | 2/032581/L64714 | 31.1 | 15.2 |
| | | | 7 | | |
| Arnica Special | Dragoco | Totowa, N.J. | 2/034591/L64106 | 28.0 | 8.5 |
| | | | 0 | | |
| Avocado | Dragoco | Totowa, N.J. | 2/034599/L64524 | 22.2 | 4.8 |
| | | | 6 | | |
| Avocado GW | Dragoco | Totowa, N.J. | 2/031170L603922 | 35.2 | 10.3 |
| Black Currant B | Dragoco | Totowa, N.J. | 2/036100/P33116 | 49.8 | 14.6 |
| | | | 6 | | |
| Black Currant Green Tea | Dragoco | Totowa, N.J. | 2/037100/P33116 | 64.0 | 17.2 |
| | | | 6 | | |
| Cabbage Rose Extract | Gattefosse | Cedex, France | 22223 | 26.1 | 11.10 |
| Calendula (Marigold) | Bell | Northbrook, | 062169 | 12.8 | 4.2 |
| | Flavors & | Il. | | | |
| | Fragrance | | | | |
| Calendula MCF 774 Hydro | Gattefosse | Cedex, France | 24243/5015 | 5.1 | 3.0 |
| Canadian Willowherb | Fytokem | Saskatoon, | 971015-P | 94.6 | 97.1 |
| (Lyophilized Powder) | | Canada | | | |
| Cemila Oleifera Extract | Gattefosse | Cedex, France | 22423 | 65.7 | 29.3 |
| Chamomile | Bio- | Hauppauge, | 980572/9831 | 4.2 | 3.1 |
| | botanica | N.J. | | | |
| Chamomile Distillate | Dragoco | Totowa, N.J. | 2/380930/L65974 | 3.3 | 2.1 |
| | | | 3 | | |
| Chamomile Special | Dragoco | Totowa, N.J. | 2/033021/694633 | 2.2 | 8.0 |
| Concombre GR 316 | Gattefosse | Cedex, France | 19190 | 2.6 | 6.8 |
| Cranberry B | Dragoco | Totowa, N.J. | 2/036600/P15193 | 10.4 | 14.6 |
| Cranberry Green Tea | Dragoco | Totowa, N.J. | 2/037600/410072 | 89.0 | 32.6 |
| | | | 3 | | |
| Cromoist Hya | Croda | Parsippany, | 7922/1 | 6.4 | 5.0 |
| | | N.J. | | | |
| Dandelion | Active | Lewisville, | S72041A/316310- | 6.1 | 2.0 |
| | organics | TX. | 11 | | |
| Dong Quai | Active | Lewisville, | S64418B/316320- | 3.2 | 2.7 |
| | organics | TX. | 11 | | |
| Echinacea Dry Aqueous | MMP Inc. | South | 7329 | 84.7 | 42.8 |
| Extract | | Plainfield, | | | |
| | | N.J. | | | |
| Garcinia | Bio- | Hauppauge, | 951283/9861 | 6.2 | 0.0 |
| | botanica | N.J. | | | |
| Ginko Biloba | Bio- | Hauppauge, | 951286/9861A | 45 | 22.3 |
| | botanica | N.J. | | | |
| Ginseng GR 471 Hydro | Gattefosse | Cedex, France | 23268/5030 | 1.9 | 1.8 |
| Glenn of Oak | Glenn | St. Paul, MN. | | 7.4 | 8.2 |
| | Corp. | | | | |
| Glenn of Orange | Glenn | St. Paul, MN. | | 5.0 | 6.5 |
| | Corp. | | | | |
| Goldenseal | Bio- | Hauppauge, | 951338/9868 | 26.0 | 0.0 |
| | botanica | N.J. | | | |
| Gotu Kola PG 5:1 | Bio- | Hauppauge, | | 9.7 | 13.5 |
| | botanica | N.J. | | | |
| Grape Extract | Gattefosse | Cedex, France | 22151 | 12.4 | 2.7 |
| Grape Seed | Active | Lewisville, | S76920B/318560- | 5.6 | 12.1 |
| | organics | TX. | 11 | | |
| Grape Seed Extract | Dragoco | Totowa, N.J. | 2/03199/P17400 | 91.1 | 95.0 |
| (powder) | | | | | |
| Grapefruit B | Dragoco | Totowa, N.J. | 2/036150/L41003 | 16.2 | 0.0 |
| | | | 13 | | |
| Grapefruit Green Tea | Dragoco | Totowa, N.J. | 2/037150/L41002 | 92.8 | 50.3 |
| | | | 11 | | |
| Green Tea | Bio- | Hauppauge, | 9945 | 85.1 | 38.7 |
| | botanica | N.J. | | | |
| Green Tea Conc. | Active- | Lewisville, | 308463/300230- | 0.0 | 4.1 |
| | organics | TX. | 94 | | |
| Green Tea Extra | Dragoco | Totowa, N.J. | 2/031598/3066 | 94.5 | 96.2 |
| (powder) | | | | | |
| Green Tea HS | Alban | Northvale, | 7114309 | 28.4 | 11.1 |
| | Muller | N.J. | | | |
| Hexaplant Richter | Chemisches | Berlin, | 732431/243 | 20.2 | 2.4 |
| | Lab | Germany | | | |
| Hibiscus Special | Dragoco | Totowa, N.J. | 2/033115/L65102 | 12.2 | 2.5 |
| | | | 8 | | |
| Hydrocotyl | Gattefosse | Cedex, France | 22842/5038 | 6.2 | 2.1 |
| Hydrolite-5 | Dragoco | Totowa, N.J. | 2/016020/27033 | 3.6 | 0.0 |
| Hydrosoy 2000 S.F. | Croda | Parsippany, | 8262 | 10.2 | 16.0 |
| | | N.J. | | | |
| Kiwi | Gattefosse | Cedex, France | 23311 | 6.5 | 0.0 |
| White Nettle | Gattefosse | Cedex, France | 22571/5040 | 4.3 | 2.5 |
| Lavender | Gattefosse | Cedex, France | 21189 | 17.0 | 14.8 |
| Lemon Extract | Gattefosse | Cedex, France | 24126 | 5.2 | 7.1 |
| Lime Blossom | Dragoco | Totowa, N.J. | 2/033093/L64404 | 98.4 | 8.7 |
| | | | 9 | | |
| Lily | Gattefosse | Cedex, France | 23410/5044 | 7.4 | 6.5 |
| Horse Chestnut | Gattefosse | Cedex, France | 22043/5046 | 22.4 | 8.2 |
| Horse Chestnut | Indena- | Uppersaddle | EG042 | 19.9 | 6.5 |
| | Intl. | River, N.J. | | | |
| German Chamomile | Indena- | Uppersaddle | EG004 | 6.7 | 8.0 |
| | Intl. | River, N.J. | | | |
| Sweet Clover | Gattefosse | Cedex, France | 23316/5051 | 12.3 | 4.3 |
| Milk Thistle | Active- | Lewisville, | S76894A/344000- | 10.0 | 4.9 |
| | organics | Tx. | 11 | | |
| Nab Willow Bark Extract | Brooks | S. Plainfield, | 28392 | 8.6 | 23.1 |
| | Indus. | N.J. | | | |
| Orange B | Dragoco | Totowa, N.J. | 2/036400/P32791 | 5.9 | 0.0 |
| | | | 1 | | |
| Orange Green Tea | Dragoco | Totowa, N.J. | 2/037400/P32791 | 93.4 | 39.6 |
| | | | 1 | | |
| Papaya B | Dragoco | Totowa, N.J. | 2/036450/L81024 | 10.4 | 0.0 |
| | | | 75 | | |
| Phytexcell Arnica | Croda | Parsippany, | 972/34656 | 42.8 | 12.6 |
| | | N.J. | | | |
| Phytexcell Mulberry | Croda | Parsippany, | 1004/34684 | 14.1 | 1.7 |
| | | N.J. | | | |
| Phytoplenolin | Bio- | Hauppauge, | 980510/9870 | 17.7 | 4.6 |
| | botanica | N.J. | | | |
| Purple Coneflower | Bio- | Hauppauge, | 951338/9852 | 23.2 | 13.6 |
| | botanica | N.J. | | | |
| Sage GW | Dragoco | Totowa, N.J. | 2/031770/L61960 | 14.7 | 5.5 |
| | | | 4 | | |
| Sage Special | Dragoco | Totowa, N.J. | 2/033291/P31250 | 7.2 | 7.6 |
| | | | 6 | | |
| Sandal Complex | Dragoco | Totowa, N.J. | 2/B04081/132360 | 11.0 | 5.5 |
| Saponaria | Gattefosse | Cedex, France | 24418/5070 | 1.2 | 0.8 |
| Sea Parsley | Phillip | Stonybrook, | 81003/R10418 | 63.0 | 16.4 |
| | Rockley | N.Y. | | | |
| Secrets D'Orange | Gattefosse | Cedex, France | 45594 | 42.3 | 19.5 |
| Soluble Wheat Protein | Croda | Parsippany, | 8018 | 11.8 | 13.0 |
| (Tritisol) | | N.J. | | | |
| St. John's Wort W/S | Dragoco | Totowa, N.J. | 2/032985/L65892 | 39.9 | 12.3 |
| | | | 6 | | |
| White Mistle Toe | Dragoco | Totowa, N.J. | 2/033141/L65332 | 27.7 | 8.3 |
| | | | 4 | | |
| White Tea 50% (powder) | Dragoco | Totowa, N.J. | 10521/C-14235 | 95.0 | 97.5 |
| Witchhazel GW | Dragoco | Totowa, N.J. | 2/031340/L65103 | 15.7 | 0.8 |
| | | | 3 | | |
| Yarrow | Bio- | Hauppauge, | 951336/9958 | 35.6 | 12.9 |
| | botanica | N.J. | | | |
| Yucca 70 | Sher-Mar | San Diego, | | 77.6 | 37.0 |
| | Entrerprises | CA. | | | |
| Yucca Extract Powder | Garuda | Santa Cruz, | 557-98226-DE- | 25.6 | 14.2 |
| 50% | | CA. | Yucext50 | | |

AST Antioxidant Status Test

Each of the above-noted compounds (test compounds) was tested to determine its antioxidant potential at three and thirty minutes as follows:

Step 1: A Total Antioxidant Status Kit from Randox Laboratories, Ltd. (United Kingdom) was obtained to measure the antioxidant potentials of the test agents in Table 1 at three and thirty minutes.

Step 2: The test agent, if in liquid form, was diluted to a 1% (v/v) solution with deionized water. If the test agent was in solid form, it was diluted to a 1% (w/v) solution with deionized water.

Step 3: From the Antioxidant Status Kit, the chromogen reagent was prepared by adding 10 milliliters of Phosphate Buffered Saline solution, pH=7.4, to a vial of chromogen 2.

Step 4: To the empty wells of a clear well plate (NUNC IMMUNO clear 96 well plate; VWR Scientific Products, Chicago, Ill.) was introduced 5 microliters of the testing agent prepared in step 2. Control wells were also prepared wherein 5 microliters of deionized water was introduced in place of the 5 microliters of testing agent. Each testing agent was tested in triplicate (n=3) with an equal number of control wells.

Step 5: To each well of the clear well plate containing the 5 microliters of testing agent (or water) was added 250 microliters of chromogen reagent as prepared under step 3 above.

Step 6: Optical density measurements of each well were taken at 600 nanometers without blanking the plate using a SPECTRAmax PLUS 384 Microplate Reader (Molecular Devices, Sunnyvale, Calif.).

Step 7: The substrate provided in the Total Antioxidant Status Kit was prepared for use by diluting 1 milliliter of the substrate with 1.5 milliliters of Phosphate Buffered Saline, pH=7.4.

Step 8: After an optical density reading was taken with testing agent (or water) plus chromogen reagent, 50 microliters of the substrate prepared in Step 7 was added to each of the sample wells.

Step 9: After three minutes (or after thirty minutes if the antioxidant potential was to be determined for thirty minutes) the sample wells of the well plate were read at 600 nanometers without blanking the plate.

Step 10: The antioxidant potential of each testing compound was calculated as follows:

% Antioxidant Potential=[(Δ_{ODWater}−Δ_{ODSample})/Δ_{ODWater})×100;

wherein

Δ_{ODWater}=OD_{Water}(Chromogen+Substrate+Water)−OD_{Water}(Chromogen+Water); and

Δ_{ODSample}=OD_{Sample}(Chromogen+Substrate+Agent)−OD_{Sample}(Chromogen+Agent)

The resulting antioxidant potentials for the testing compounds at three and thirty minutes are set forth in Table 1.

### EXAMPLE 2

In this Example, two botanical extracts which exhibited high antioxidant potentials at three and thirty minutes, as determined in Example 1, were evaluated for their ability to improve the mildness of a commercially available wet wipe solution. Specifically, the ability of the botanical extracts to reduce the level of skin irritation in an EPIDERM skin culture as determined by measuring levels of an inflammatory mediator (interleukin-1 alpha, (herein IL-1 alpha)) in the culture media underlying the tissue was studied.

EPIDERM skin cultures (EPIDERM EPI-200, MatTek Corp., Ashland, Mass.), which are a cornified, air-interfaced human skin cultures with multiple layers of progressively differentiated keratinocytes that resemble human epidermis, were utilized as an in vitro skin irritation model.

A first testing sample was prepared by making a 0.5% (w/v) sample of Green Tea Extra (Dragoco, Totowa, N.J.) in the expressed wet wipe solution. A second testing sample was prepared by making a 0.5% (w/v) sample of Grape Seed Extract (Dragoco, Totowa, N.J.) in the expressed wet wipe solution. Experiments were conducted in six well plates in which EPIDERM skin cultures (diameter of the culture insert was 0.9 centimeters) were placed in wells in which each well contained one milliliter of culture media (EPI-100 Asy/Assay Medium, MatTek Corp, Ashland, Mass.). The EPIDERM skin cultures were incubated in a 37° C., 5% carbon dioxide incubator for sixty minutes. After incubation, the media underlying the EPIDERM skin cultures was aspirated off and replaced with 1 milliliter of fresh pre-warmed media. If any media was found at the surface of the culture, it was carefully removed using a Gilson Pipetman®. Then, a 15 microliter aliquot of test sample (or deionized water control) was applied to the surface of the EPIDERM skin culture and the skin cultures incubated for 8 hours in a 37° C., 5% carbon dioxide incubator.

Skin irritation induced by the solution with and without the Green Tea Extra or the Grape Seed Extract was determined by measuring the amount of the inflammatory mediator, IL-1 alpha, in the culture media underlying the skin cultures. Interleukin-1 alpha can be quantified using an Interleukin-1 alpha Quantikine Kit available from R&D Systems (Minneapolis, Minn.). Interleukin-1 alpha measurements are converted to Log₁₀ for each of the treatments and the averages for each treatment calculated. The percent mean reduction of IL-1 alpha was calculated as follows:

% mean reduction of IL-1 alpha=100×[((wet wipe solution)result−(test sample)result)/((wet wipe solution)result−(water)result)];

wherein

(test sample) result=the measured amount of IL-1 alpha from a treatment with expressed wet wipe solution and test botanical;

(wet wipe solution) result=the measured amount of IL-1 alpha from a treatment with expressed wet wipe solution; and

(water) result=the measured amount of IL-1a from a treatment with deionized water.

The greater the percent mean reduction of IL-1 alpha, the more effective a test sample is at reducing skin irritation of the expressed wet wipe solution and improving the mildness of the solution. Results of the mean reduction of IL-1 alpha were analyzed to identify outlier results. The EPIDERM skin culture can be variable with the variability attributed to differences in the culture, variation in the application of treatment, and other factors. A statistical analysis technique was applied to identify when a result abnormally deviated from the rest of the data set. The irritation values were first converted to Log₁₀ in order to make them Gaussian. After conversion, the values were analyzed for high or low value outliers. The statistical analysis used to identify outliers is described on page 460 of “Statistical Methods in Research and Production” by Owen L. Davies et al., Published by Longman Group Limited, fourth revised edition, 1984, and is hereby incorporated by reference. The reduction in IL-1 alpha was measured and the results are shown in Table 2:

TABLE 2

| | Mean Reduction Interleukin-1 |
|---|---|
| Test Composition | Alpha 8 Hours |
| Green Tea Extra (0.5%) | 55% (5 replicates) |
| Grape Seed Extract (0.5%) | 29% (5 replicates) |

The data indicates that the inclusion of Green Tea Extra or Grape Seed Extract in an expressed wet wipe solution reduces the mean IL-1 alpha skin irritation score and increases the mildness of the wet wipe solution.

In view of the above, it will be seen that the several objects of the invention are achieved. As various changes could be made in the above-described products and methods without departing from the scope of the invention, it is intended that all matter contained in the above description be interpreted as illustrative and not in a limiting sense.

What is claimed is:

1. A wipe for improving skin health comprising a fibrous wipe substrate and an antioxidant agent for reducing oxidative-induced skin damage, the antioxidant agent having an antioxidant potential at three minutes of at least about 25%.

2. The wipe as set forth in claim 1 wherein the antioxidant agent for reducing oxidative-induced skin damage has an antioxidant potential at three minutes of at least about 25% as determined by a AST Antioxidant Status Test.

3. The wipe as set forth in claim 1 wherein the agent is selected from the group consisting of Apple Green Tea, Arkin Special, Arnica Special, Avocado GW, Black Currant B, Black Currant Green Tea, Cabbage Rose Extract, Canadian Willowherb, Cemila Oleifera Extract, Cranberry Green Tea, Echinacea Dry Aqueous Extract, Goldenseal, Grape Seed Extract, Grapefruit Green Tea, Green Tea, Green Tea Extra, Green Tea HS, Lime Blossom, Orange Green Tea, Phytexcell Arnica, Sea Parsley, Secrets D' Orange, St. John's Wort W/S, White Mistle Toe, White Tea 50%, Yarrow, Yucca 70, Yucca Extract Powder (50%), and combinations thereof.

4. The wipe as set forth in claim 1 wherein the agent is selected from the group consisting of Apple Green Tea, Canadian Willowherb, Cranberry Green Tea, Echinacea Dry Aqueous Extract, Grape Seed Extract, Grapefruit Green Tea, Green Tea, Green Tea Extra, Lime Blossom, Orange Green Tea, White Tea 50%, Yucca 70, and combinations thereof.

5. The wipe as set forth in claim 1 wherein the antioxidant agent has an antioxidant potential at three minutes of at least about 50%.

6. The wipe as set forth in claim 1 wherein the antioxidant agent has an antioxidant potential at three minutes of at least about 75%.

7. The wipe as set forth in claim 1 wherein the wipe is a dry wipe and the agent is present in an amount of from about 0.01% (by weight of the treated substrate) to about 50% (by weight of the treated substrate).

8. The wipe as set forth in claim 1 wherein the wipe is a wet wipe and further comprises a liquid formulation, said agent being present in an amount of from about 0.01% (by total weight of the liquid formulation) to about 50% (by total weight of the liquid formulation).

9. A wipe for improving skin health comprising a fibrous wipe substrate and an antioxidant agent for reducing oxidative-induced skin damage, the antioxidant agent having an antioxidant potential at thirty minutes of at least about 20%.

10. The wipe as set forth in claim 9 wherein the antioxidant agent for reducing oxidative-induced skin damage has an antioxidant potential at thirty minutes of at least about 20% as determined by a AST Antioxidant Status Test.

11. The wipe as set forth in claim 9 wherein the agent is selected from the group consisting of Apple Green Tea, Canadian Willowherb, Cemila Oleifera Extract, Cranberry Green Tea, Echinacea Dry Aqueous Extract, Ginko Biloba, Grape Seed Extract, Grapefruit Green Tea, Green Tea, Green Tea Extra, Nab Willow Bark, Orange Green Tea, White Tea 50%, Yucca 70, and combinations thereof.

12. The wipe as set forth in claim 9 wherein the agent is selected from the group consisting of Grape Seed Extract, Green Tea Extra, White Tea 50%, and combinations thereof.

13. The wipe as set forth in claim 9 wherein the antioxidant agent has an antioxidant potential at thirty minutes of at least about 50%.

14. The wipe as set forth in claim 9 wherein the antioxidant agent has an antioxidant potential at thirty minutes of at least about 75%.

15. The wipe as set forth in claim 9 wherein the wipe is a dry wipe and the agent is present in an amount of from about 0.01% (by weight of the treated substrate) to about 50% (by weight of the treated substrate).

16. The wipe as set forth in claim 1 wherein the wipe is a wet wipe and further comprises a liquid formulation, said agent being present in an amount of from about 0.01% (by total weight of the liquid formulation) to about 50% (by total weight of the liquid formulation).

17. A wipe for improving skin health comprising a fibrous wipe substrate and an antioxidant agent for reducing oxidative-induced skin damage, the antioxidant agent having an antioxidant potential at three minutes of at least about 75% and an antioxidant potential at thirty minutes of at least about 75%.

18. The wipe as set forth in claim 17 wherein the agent is selected from the group consisting of Grape Seed Extract, Canadian Willowherb, Green Tea Extra, White Tea 50%, and combinations thereof.

19. The wipe as set forth in claim 17 wherein the wipe is a dry wipe and the agent is present in an amount of from about 0.01% (by weight of the treated substrate) to about 50% (by weight of the treated substrate).

20. The wipe as set forth in claim 17 wherein the wipe is a wet wipe and further comprises a liquid formulation, said agent being present in an amount of from about 0.01% (by total weight of the liquid formulation) to about 50% (by total weight of the liquid formulation).

21. A product for improving skin health comprising an absorbent substrate and an antioxidant agent for reducing oxidative-induced skin damage, the antioxidant agent having an antioxidant potential at three minutes of at least about 25%.

22. The product as set forth in claim 21 wherein the antioxidant agent for reducing oxidative-induced skin damage has an antioxidant potential at three minutes of at least about 25% as determined by a AST Antioxidant Status Test.

23. The product as set forth in claim 21 wherein the agent is selected from the group consisting of Apple Green Tea, Arnica Special, Avocado GW, Black Currant B, Black Currant Green Tea, Cabbage Rose Extract, Canadian Willowherb, Cemila Oleifera Extraxt, Cranberry Green Tea, Echinacea Dry Aqueous Extract, Goldenseal, Grape Seed Extract, Grapefruit Green Tea, Green Tea, Green Tea Extra, Green Tea HS, Lime Blossom, Orange Green Tea, Sea Parsley, St. John's Wort W/S, White Mistle Toe, White Tea 50%, Yarrow, Yucca 70, Yucca Extract Powder (50%), and combinations thereof.

24. The product as set forth in claim 21 wherein the agent is selected from the group consisting of Apple Green Tea, Canadian Willowherb, Cranberry Green Tea, Echinacea Dry Aqueous Extract, Grape Seed Extract, Grapefruit Green Tea, Green Tea, Green Tea Extra, Lime Blossom, Orange Green Tea, White Tea 50%, Yucca 70, and combinations thereof.

25. The product as set forth in claim 21 wherein the agent is present in an amount of from about 0.01% (by weight of the treated product) to about 50% (by weight of the treated product).

26. The product as set forth in claim 21 wherein the product is selected from the group consisting of a diaper, training pants, adult incontinence garments, feminine napkins, paper towels, tampons, interlabial pads, facial tissue, wound management products, and bath tissue.

27. A product for improving skin health comprising an absorbent substrate and an antioxidant agent for reducing oxidative induced skin damage, the antioxidant agent having an antioxidant potential at thirty minutes of at least about 20%.

28. The product as set forth in claim 27 wherein the antioxidant agent for reducing oxidative induced skin damage has an antioxidant potential at thirty minutes of at least about 20% as determined by a AST Antioxidant Status Test.

29. The product as set forth in claim 27 wherein the agent is selected from the group consisting of Apple Green Tea, Canadian Willowherb, Cemila Oleifera Extract, Cranberry Green Tea, Echinacea Dry Aqueous Extract, Ginko Biloba, Grape Seed Extract, Grapefruit Green Tea, Green Tea, Green Tea Extra, Nab Willow Bark, Orange Green Tea, White Tea 50%, Yucca 70, and combinations thereof.

30. The product as set forth in claim 27 wherein the agent is selected from the group consisting of Grape Seed Extract, Green Tea Extra, White Tea 50%, and combinations thereof.

31. The product as set forth in claim 27 wherein the agent is present in an amount of from about 0.01% (by weight of the treated product) to about 50% (by weight of the treated product).

32. The product as set forth in claim 27 wherein the product is selected from the group consisting of a diaper, training pants, adult incontinence garments, feminine napkins, paper towels, tampons, interlabial pads, facial tissue, wound management products, and bath tissue.

33. A product for improving skin health comprising an absorbent substrate and an antioxidant agent for reducing oxidative-induced skin damage, the antioxidant agent having an antioxidant potential at three minutes of at least about 75% and an antioxidant potential at thirty minutes of at least about 75%.

34. A method for controlling oxidative induced skin damage to improve skin health, the method comprising contacting a fibrous wipe substrate with the skin surface, the fibrous wipe substrate comprising an antioxidant agent for reducing oxidative-induced skin damage, the antioxidant agent having an antioxidant potential at three minutes of at least about 25%.

35. The method as set forth in claim 34 wherein the wipe is a wet wipe and further comprises a liquid formulation, the antioxidant agent being present in an amount of from about 0.01% (by total weight of the liquid formulation) to about 50% (by total weight of the liquid formulation).

36. A wet wipe for improving skin health comprising a fibrous wipe substrate, a liquid formulation, and an antioxidant agent for reducing oxidative-induced skin damage, the antioxidant agent having an antioxidant potential at three minutes of at least about 25% and being present in an amount of from about 0.01% (by total weight of the liquid formulation) to about 50% (by total weight of the liquid formulation), and the liquid formulation being selected from the group consisting of a solution, a suspension, and an emulsion.

37. The wet wipe as set forth in claim 36 wherein the liquid formulation is an emulsion selected from the group consisting of an oil-in-water emulsion and a water-in-oil emulsion.

38. The wet wipe as set forth in claim 36 wherein the antioxidant agent is encapsulated.
